(19) United States
(12) Patent Application Publication
(10) Pub. No.: US 2019/0290673 A1
(43) Pub. Date: Sep. 26, 2019

(54) COMPOSITIONS AND METHODS FOR THE TREATMENT OF HBV INFECTION

(71) Applicant: Spring Bank Pharmaceuticals, Inc., Hopkinton, MA (US)

(72) Inventors: Radhakrishnan P. Iyer, Shrewsbury, MA (US); Rayomand H. Gimi, Chelmsford, MA (US)

(21) Appl. No.: 16/344,163

(22) PCT Filed: Oct. 24, 2017
(86) PCT No.: PCT/US2017/058047

§ 371 Date: Apr. 23, 2019

Related U.S. Application Data
(60) Provisional application No. 62/412,058, filed on Oct. 24, 2016.

(51) Int. Cl. A61K 31/7076; A61K 9/00; A61P 31/20

(52) U.S. Cl. CPC A61K 31/7076; A61P 31/20; A61K 9/0053

(57) ABSTRACT

This invention relates to methods useful in the treatment of a hepatitis infection.

### CROSS REFERENCE TO RELATED APPLICATION

This application is a United States National Phase Application under 35 U.S.C. § 371 of International Application Number PCT/US2017/058047 filed Oct. 24, 2017, which claims the benefit of and priority to U.S. Provisional Patent Application No. 62/412,058 filed Oct. 24, 2016, which is hereby incorporated by reference herein in its entirety.

### FIELD OF INVENTION

This invention relates to compositions and methods useful in the treatment of a viral infection (e.g., a Hepatitis B infection).

### BACKGROUND OF INVENTION

Chronic infection with hepatitis B virus (HBV) is a major public health problem and is responsible for approximately 1.2 million deaths per year worldwide due to HBV-associated liver diseases, such as hepatic cirrhosis, and hepatocellular carcinoma (HCC) (Levanchy, D. J Viral Hepatol (2004) 11:97-107). It is estimated that more than 2 billion people have serological evidence of previous or current HBV infection, and that over 350 million individuals are chronic carriers of HBV (Levanchy, D. J Viral Hepatol (2004) 11:97-107; Kwon H., Lok. A. S. Nat Rev Gastroenterol Hepatol (2011) 8:275-284). Although safe and effective prophylactic vaccines against HBV are available, improvements in therapeutics for treatment of chronic HBV infection are still urgently needed. Current antiviral therapies for chronic hepatitis B (CHB) are limited, and include nucleoside and nucleotide analogs and interferon (IFN) treatment. While administration of nucleosides and nucleotides may reduce viral load and improve the long-term outcome of CHB, prolonged use rarely leads to a cure. Only 2-3% of treated patients per year experience a loss of measurable biomarkers of HBV infection, namely durable loss of HBV surface antigen (HBsAg) and seroconversion to antibodies against HBsAg (anti-HBs) (Kwon H., Lok. A. S. Nat Rev Gastroenterol Hepatol (2011) 8:275-284). Long-term IFN administration is also associated with treatment-limiting adverse effects and variability in treatment response, and while the rate of durable HBsAg loss is higher than with nucleoside and nucleotide analogs, it still only occurs in less than 10% of patients.

A major obstacle for treatment of HBV infection relates to the emergence of drug resistant variants that occurs upon extended use of currently available nucleoside and nucleotide analogs, many of which target the viral DNA polymerase. In addition, current treatments require persistent and long-term use, which often results in unwarranted side effects and the risk of relapse upon treatment discontinuation. Accordingly, there is a critical need for a new generation of therapies to combat HBV infection.

### SUMMARY OF INVENTION

Described herein are compounds and compositions for the treatment of a viral infection. In one aspect, the present invention features a method of treating a subject infected with the Hepatitis B virus with a salt form of a compound of Formula (I), wherein the compound is selected from:

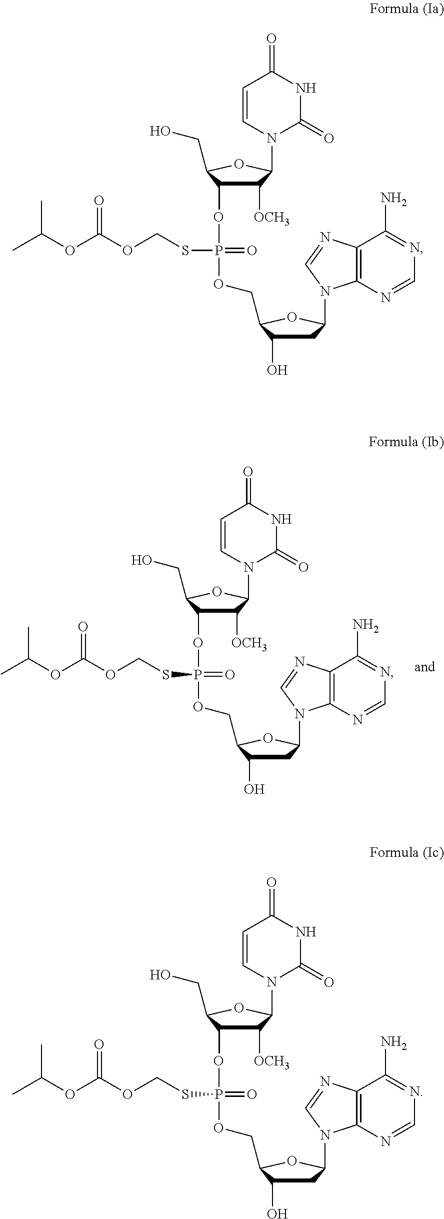

In some embodiments, the salt form of a compound of Formula (I) is a tartrate salt.

In some embodiments, the subject is administered a composition comprising a mixture of compounds of Formula (I) (e.g., as described by Formula (Ia)). In some embodiments, the composition comprises a mixture of Formula (Ib) and Formula (Ic). In some embodiments, the mixture comprises a ratio of Formula (Ib) to Formula (Ic) of about 1:1 (e.g., a racemic mixture). In some embodiments, the mixture comprises a ratio of Formula (Ib) to Formula (Ic) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (Ic) to Formula (Ib) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the composition comprises Formula (Ib) and comprises less than about 5% of Formula (Ic), e.g., less than about 4%, less than about 3%, less than about 2%, less than about 1%, less than about 0.5%, or less than about 0.1% of Formula (Ic), or is substantially free of Formula (Ic). In some embodiments, the composition comprises Formula (Ic) and comprises less than about 5% of Formula (Ib), e.g., less than about 4%, less than about 3%, less than about 2%, less than about 1%, less than about 0.5%, or less than about 0.1% of Formula (Ib), or is substantially free of Formula (Ib).

In some embodiments, the compound is selected from:

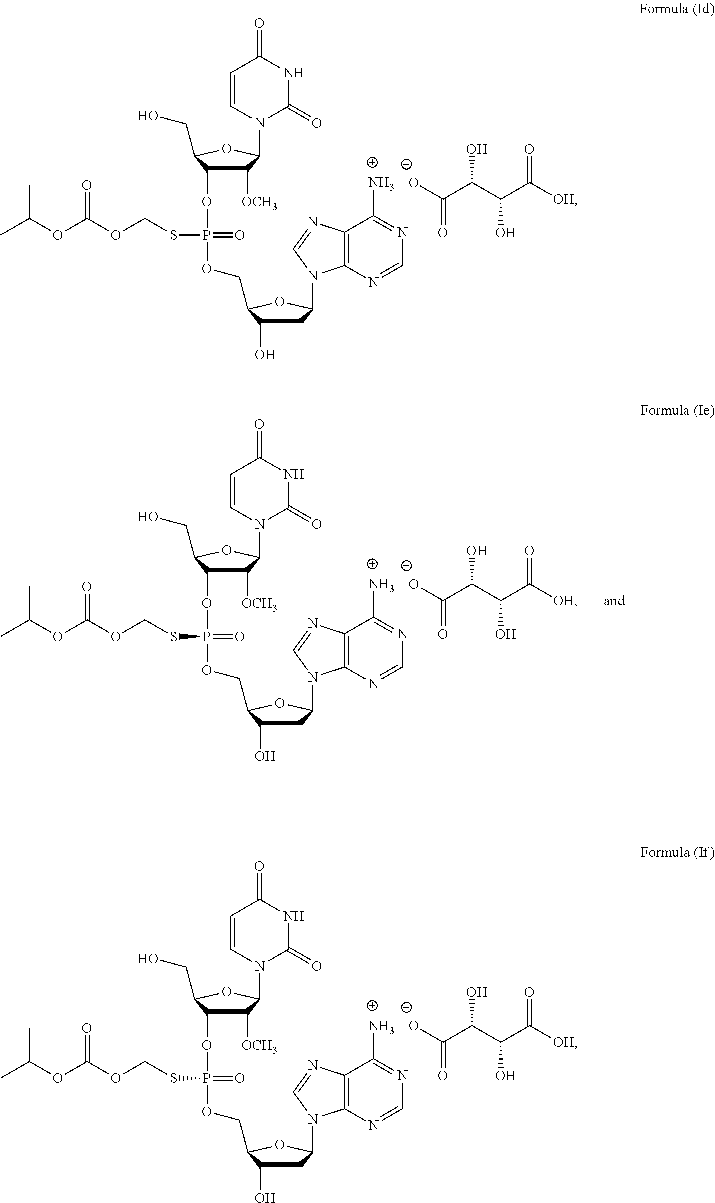

In some embodiments, the subject is administered a composition comprising a mixture of compounds of Formula (I) (e.g., as described by Formula (Id)). In some embodiments, the composition comprises a mixture of Formula (Ie) and Formula (If). In some embodiments, the mixture comprises a ratio of Formula (Ie) to Formula (If) of about 1:1 (e.g., a racemic mixture). In some embodiments, the mixture comprises a ratio of Formula (Ie) to Formula (If) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (If) to Formula (Ie) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the composition comprises Formula (Ie) and comprises less than about 5% of Formula (If), e.g., less than about 4%, less than about 3%, less than about 2%, less than about 1%, less than about 0.5%, or less than about 0.1% of Formula (If), or is substantially free of Formula (If). In some embodiments, the composition comprises Formula (If) and comprises less than about 5% of Formula (Ie), e.g., less than about 4%, less than about 3%, less than about 2%, less than about 1%, less than about 0.5%, or less than about 0.1% of Formula (Ie), or is substantially free of Formula (Ie).

In some embodiments, the composition is administered orally. In some embodiments, the composition is administered parenterally (e.g., intraperitoneally).

In some embodiments, the subject is a mammal. In some embodiments, the subject is a human. In some embodiments the subject is a non-human animal, e.g., a mouse or a woodchuck (e.g., Eastern woodchuck).

In some embodiments, the method comprises daily administration of said compound or a composition thereof. In some embodiments, the administration is once daily. In some embodiments, the administration is greater than once daily, e.g., twice daily, three times daily, four times daily.

In some embodiments, the method comprises administration of said compound or a composition thereof at a frequency less than once a day, e.g., once every 36 hours, once every other day, or once a week.

In some embodiments, the dosage of a compound of Formula (I) (e.g., a compound of Formula (Ia), Formula (Ib), Formula (Ic), Formula (Id), Formula (Ie), or Formula (If)) comprises about 0.5 mg/kg to about 100 mg/kg. In some embodiments, the dosage comprises about 0.5 mg/kg to about 95 mg/kg, about 90 mg/kg, about 85 mg/kg, about 80 mg/kg, about 75 mg/kg, about 70 mg/kg, about 65 mg/kg about 60 mg/kg, about 55 mg/kg, about 50 mg/kg, about 45 mg/kg, about 40 mg/kg, about 35 mg/kg, about 30 mg/kg, about 25 mg/kg, about 20 mg/kg, about 15 mg/kg, or about 10 mg/kg. In some embodiments, the dosage comprises about 0.5 mg/kg to about 50 mg/kg. In some embodiments, the dosage comprises about 0.5 mg/kg to about 40 mg/kg.

In some embodiments, the dosage comprises greater than about 0.5 mg/kg, e.g., about 1.0 mg/kg, about 1.5 mg/kg, about 2 mg/kg, about 3 mg/kg, about 4 mg/kg, about 5 mg/kg, about 10 mg/kg about 15 mg/kg, about 20 mg/kg, about 25 mg/kg, about 30 mg/kg, about 35 mg/kg, about 40 mg/kg, about 50 mg/kg, about 55 mg/kg, about 60 mg/kg, about 65 mg/kg, about 70 mg/kg, about 75 mg/kg, about 80 mg/kg, about 85 mg/kg, or about 90 mg/kg up to about 100 mg/kg. In some embodiments, the dosage comprises about 5 mg/kg to about 50 mg/kg. In some embodiments, the dosage comprises about 10 mg/kg to about 50 mg/kg. In some embodiments, the dosage comprises about 15 mg/kg to about 50 mg/kg.

In some embodiments, the dosage comprises a liquid or a solid dosage form. In some embodiments, the liquid dosage form comprises a suspension, a solution, a linctus, an emulsion, a drink, an elixir, or a syrup. In some embodiments, the solid dosage form comprises a capsule, tablet, dragée, or powder. In some embodiments, the liquid or solid dosage form is orally administered. In some embodiments, the liquid or solid form is parenterally (e.g., intraperitoneally) administered.

In some embodiments, the method further comprises the administration of an additional agent. In some embodiments, the method further comprises the administration of a therapeutically effective amount of an additional agent. In some embodiments, the additional agent is an antiviral agent or an anticancer agent. In some embodiments, the antiviral agent comprises an interferon, a nucleoside analog, a non-nucleoside antiviral, or a non-interferon immune enhancer. In some embodiments, the interferon comprises interferon alfa-2a, interferon alfa-2b, interferon alfa-n1, interferon alfacon-1, or a pegylated interferon (e.g., peginterferon alfa-2a, peginterferon alfa-2b). In some embodiments, the nucleoside analog comprises lamivudine, adefovir dipivoxil, entecavir, telbivudine, clevudine, ribavarin, tenofovir, tenofovir dipivoxil, tenofovir alafenamide, besifovir, or AGX-1009. In some embodiments, the antiviral agent is entecavir. In some embodiments, the antiviral compound comprises NOV-225, BAM 205, Myrcludex B, ARC-520, BAY 41-4109, REP 9AC, Alinia (nitazoxanide), Dd-RNAi, NVR-121 (NVR 3-778), BSBI-25, NVP-018, TKM-HBV, or ALN-HBV. In some embodiments, the non-interferon immune enhancer comprises zadaxin (thymosin alpha-1), GS-4774, CYT107 (interleukin-7), Dv-601, HBV core antigen vaccine, or GS-9620. In some embodiments, the antiviral agent is a capsid inhibitor, an entry inhibitor, a secretion inhibitor, a microRNA, an antisense RNA agent, an RNAi agent, or other agent designed to inhibit viral RNA. In some embodiments, the anticancer agent is selected from methotrexate, 5-fluorouracil, doxorubicin, vincristine, bleomycin, vinblastine, dacarbazine, toposide, cisplatin, epirubicin, and sorafenib tosylate.

In some embodiments, in a method described herein, the subject is treatment naïve. In some embodiments, the subject has previously been treated for HBV infection. In some embodiments, the previous treatment for HBV infection has failed. In some embodiments, the subject has relapsed.

In some embodiments, the subject has been previously been treated with an anti-HBV agent other than a compound of Formula (I) (e.g., an interferon, ribavirin) and is suffering from a relapsed HBV infection.

In some embodiments, the methods described herein further comprise analyzing or receiving analysis of the body weight and temperature of the subject at least once a week until the end of treatment.

In some embodiments, the methods described herein further comprise analyzing or receiving analysis of a blood sample from the subject at least once prior to the end of treatment. In some embodiments, the blood sample is analyzed for viral load or antigen level, e.g., relative to a reference standard. In some embodiments, the antigen level comprises the level of e antigen (e.g., HBeAg), surface antigen (e.g., HBsAg), or core antigen (e.g., HBCrAg), e.g., relative to a reference standard. In some embodiments, the blood sample is analyzed for the expression level of interferon (e.g., interferon alfa or interferon beta), an interferon stimulating protein (e.g., ISG15, CXCL10, OAS 1), or other cytokine, e.g., relative to a reference standard.

In some embodiments, the methods described herein further comprise analyzing or receiving analysis of a liver biopsy specimen from the subject at least once prior to the end of treatment. In some embodiments, the liver biopsy specimen is analyzed for the level of viral DNA, viral RNA, a viral antigen, or cccDNA, e.g., relative to a reference standard. In some embodiments, the liver biopsy specimen is analyzed for the expression level of interferon (e.g., interferon alfa or interferon beta), an interferon stimulating protein (e.g., ISG15, CXCL10, OAS 1), or other cytokine, e.g., relative to a reference standard. In some embodiments, the liver biopsy specimen is analyzed for the expression level of a pattern recognition receptor, e.g., relative to a reference standard. Exemplary pattern recognition receptors include RIG-I, NOD2, STING, and others. In some embodiments, the liver biopsy specimen is analyzed for the reduction of liver inflammation, necrosis, steatosis, or fibrosis, e.g., relative to a reference standard.

### BRIEF DESCRIPTION OF THE DRAWINGS

FIG. 1 is a graph showing the distribution of serum HBeAg pre-screened values before initiation of treatment described in Example 2.

FIGS. 2A-2B are graphs showing the effect of exemplary compounds on liver HBV DNA levels using Southern blot hybridization (FIG. 2A) and semi-quantitative PCR (FIG. 2B). ***P≤0.001 using one-way analysis of variance compared to vehicle group. ##P≤0.01 using unpaired two-tail t test compared to Formula (Ie).

FIG. 3 is an autoradiograph of Southern blot hybridization specific for HBV DNA based on the study described in Example 2. The top band is the transgenic mouse signal.

FIGS. 4A-4B are graphs showing the effect of exemplary agents on serum levels of HBeAg (FIG. 4A) and HBsAg (FIG. 4B) in the study described in Example 2. As shown, there was no statistical significance compared to vehicle values.

FIG. 5 is a graph showing the effect of exemplary compounds on whole body weight change in the study described in Example 2. As shown, there was no statistical significance compared to vehicle values.

### DETAILED DESCRIPTION OF THE INVENTION

The present invention relates to compounds and particular salt forms (e.g., tartrate salts thereof) and methods of treating a subject infected with the Hepatitis B virus, the method comprising administration of a compound of Formula (I) (e.g., Formula (Ia), Formula (Ib), Formula (Ic), Formula (Id), Formula (Ie), or Formula (If)) or a composition thereof.

### Definitions

As used herein, the articles “a” and “an” refer to one or to more than one (e.g., to at least one) of the grammatical object of the article.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the term “acquire” or “acquiring” as the terms are used herein, refer to obtaining possession of a physical entity (e.g., a sample, e.g., blood sample or liver biopsy specimen), or a value, e.g., a numerical value, by “directly acquiring” or “indirectly acquiring” the physical entity or value. “Directly acquiring” means performing a process (e.g., an analytical method) to obtain the physical entity or value. “Indirectly acquiring” refers to receiving the physical entity or value from another party or source (e.g., a third party laboratory that directly acquired the physical entity or value). Directly acquiring a value includes performing a process that includes a physical change in a sample or another substance, e.g., performing an analytical process which includes a physical change in a substance, e.g., a sample, performing an analytical method, e.g., a method as described herein, e.g., by sample analysis of bodily fluid, such as blood by, e.g., mass spectroscopy (e.g. LC-MS), or PCR (e.g., RT-PCR).

As used herein, an amount of a compound or substance effective to treat a disorder (e.g., a disorder described herein), “therapeutically effective amount,” “effective amount” or “effective course” refers to an amount of the compound, substance, or composition which is effective, upon single or multiple dose administration(s) to a subject, in treating a subject, or in curing, alleviating, relieving or improving a subject with a disorder (e.g., an HBV infection) beyond that expected in the absence of such treatment.

As used herein, the terms “prevent” or “preventing” as used in the context of a disorder or disease, refer to administration of an agent to a subject, e.g., the administration of a compound of the present invention (e.g., compound of Formula (I)) to a subject, such that the onset of at least one symptom of the disorder or disease is delayed as compared to what would be seen in the absence of administration of said agent.

As used herein, the term “prodrug” refers to a compound which, when metabolized (e.g., in vivo or in vitro), yields an active compound. In some embodiments, the prodrug may be inactive, or possess less activity that the free drug, but may provide advantageous handling, administration, or metabolic properties. Exemplary prodrug moieties of the present invention may be linked to the free drug through the hydroxyl, amino, phosphate, or phosphorothioate backbone of the nucleotide, and may comprise an ester, a carbamate, a carbonyl, a thioester, amide, isocyanate, urea, thiourea, or other physiologically acceptable metabolically labile moiety. In some embodiments, a prodrug is activated through enzymatic hydrolysis.

As used herein, the term “reference standard” refers to a standardized level or standardized treatment that is used as basis for comparison. In some embodiments, the reference standard is an accepted, well known, or well characterized standard or treatment in the art. In some embodiments, the reference standard describes an outcome of a method described herein. In some embodiments, the reference standard describes a level of a marker (e.g., viral load, viral DNA, viral RNA, viral antigen, cccDNA, interferon, interferon stimulating protein, or a pattern recognition receptor (e.g., RIG-I, NOD2, STING)) in a subject or a sample, e.g., prior to initiation of treatment, e.g., with a compound or composition described herein. In some embodiments, the reference standard describes a measure of the presence of, progression of, or severity of a disease or the symptoms thereof, e.g., prior to initiation of treatment, e.g., with a compound or composition described herein.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dogs, cats, cows, pigs, etc.

As used herein, the terms “treat” or “treating” a subject having a disorder or disease refer to subjecting the subject to a regimen, e.g., the administration of a compound of Formula (I), such that at least one symptom of the disorder or disease is cured, healed, alleviated, relieved, altered, remedied, ameliorated, or improved. Treating includes administering an amount effective to alleviate, relieve, alter, remedy, ameliorate, improve or affect the disorder or disease, or the symptoms of the disorder or disease. The treatment may inhibit deterioration or worsening of a symptom of a disorder or disease.

Numerous ranges, e.g., ranges for the amount of a drug administered per day, are provided herein. In some embodiments, the range includes both endpoints. In other embodiments, the range excludes one or both endpoints. By way of example, the range can exclude the lower endpoint. Thus, in such an embodiment, a range of 250 to 400 mg/day, excluding the lower endpoint, would cover an amount greater than 250 that is less than or equal to 400 mg/day.

### Compounds and Therapeutic Agents

The present invention features methods for treatment of a subject infected with HBV comprising administration of a salt form of a compound of Formula (I) (e.g., a tartrate salt form) or composition thereof. The compound of Formula (I) is a prodrug in which the active agent is Formula (II), which may be described by any one of Formula (IIa), Formula (IIb), and Formula (IIc), or a combination thereof:

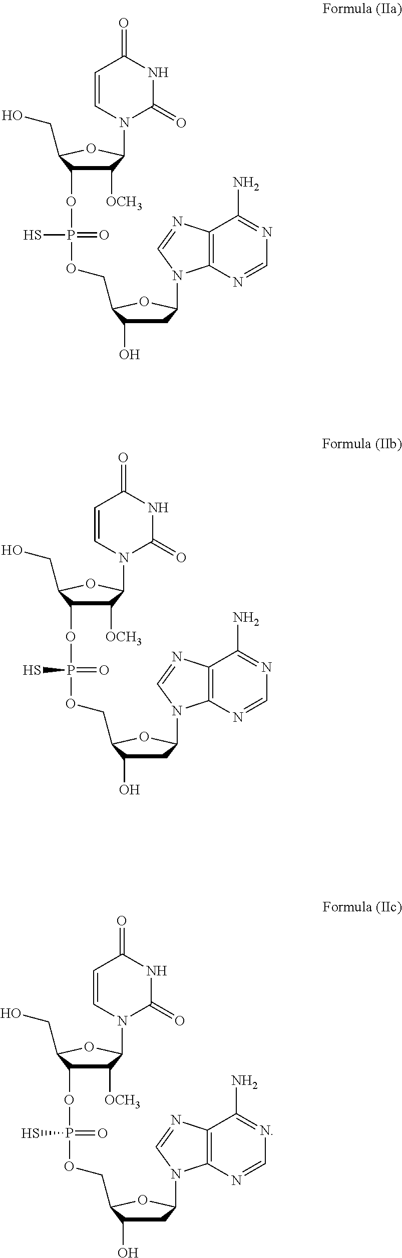

In some embodiments, the prodrug is a compound of Formula (I) (e.g., Formula (Ia), Formula (Ib), Formula (Ic), Formula (Id), Formula (Ie), or Formula (If)) and is activated through enzymatic hydrolysis.

In certain embodiments, the compound of Formula (I) is a salt form and may be described by any one of Formula (Ia), Formula (Ib), Formula (Ic), or a combination thereof:

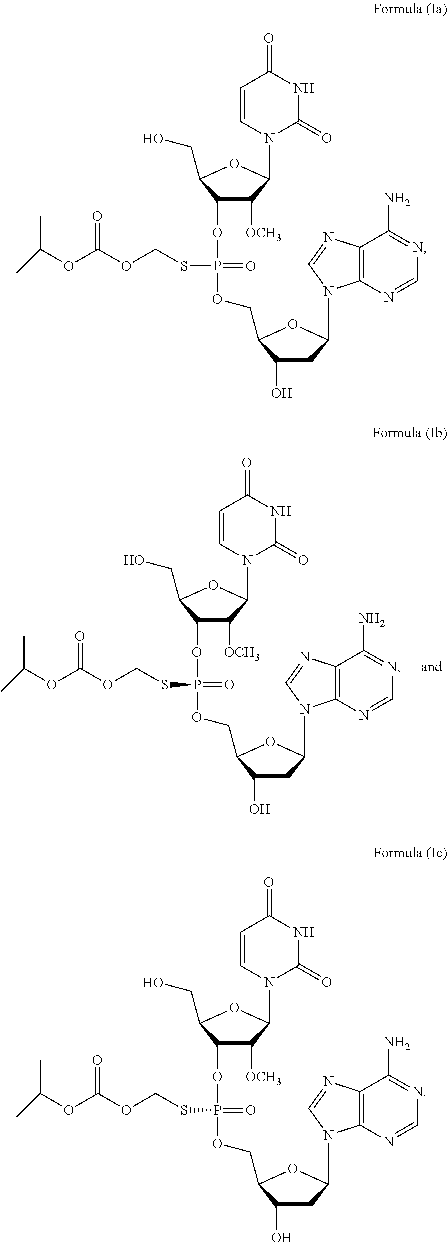

In some embodiments, the salt form of a compound of Formula (I) is a tartrate salt.

In some embodiments, the compound of Formula (I) is selected from:

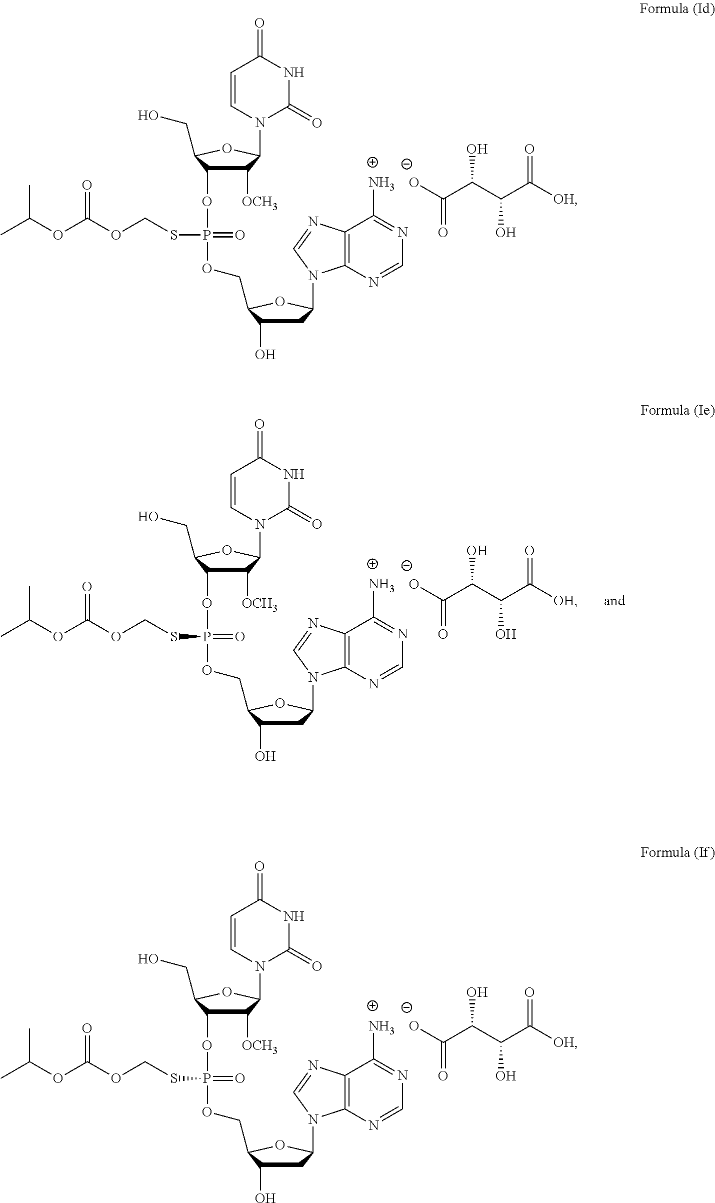

Formula (II) and its prodrug Formula (I) are small molecule nucleic acid hybrid (dinucleotide) compounds that combine both antiviral and immune modulating activities. The latter activity mediates controlled apoptosis of virus-infected hepatocytes via stimulation of the innate immune response, similar to what is also achieved by IFN-α therapy in HBV-infected patients.

Without wishing to be bound by theory, the mechanism of action of Formula (II) and its prodrug Formula (I) may be dissected into two components. The first component entails the host immune stimulating activity of Formula (II), which induces endogenous IFNs via the activation of viral sensor proteins, e.g., retinoic acid-inducible gene 1 (RIG-I) and nucleotide-binding oligomerization domain-containing protein 2 (NOD2) (Takeuchi, O. and Akira S. Cell (2010) 140:805-820; Sato, S. et al. Immunity (2015) 42:123-132; Sabbah, A. et al. Nat Immunol (2009) 10:1073-1080). Activation may occur by binding of Formula (II) to the RIG-I/NOD2 proteins at their nucleotide binding domain. The RIG-I and NOD2 proteins are located in the cytosol of cells, including hepatocytes, and usually recognize signature patterns of foreign nucleic acids such as the pathogen associated molecular pattern (PAMP). Once PAMP within viral RNA or DNA is recognized, RIG-I and NOD2 may become activated and trigger the IFN signaling cascade that then results in IFN and interferon-stimulated gene (ISG) production and induction of an antiviral state in cells. In the case of HBV, the PAMP is believed to be the pre-genomic RNA which has a significant double-stranded RNA structure known as epsilon structure.

The second component of the mechanism of action of Formula (II) and its prodrug Formula (I) involves its direct antiviral activity, which inhibits the synthesis of viral nucleic acids by steric blockage of the viral polymerase. The block may be achieved by interaction Formula (II) with RIG-I and NOD2 as described above that then in turn may prevent the polymerase enzyme from engaging with the viral nucleic acid template for replication (i.e, HBV pre-genomic RNA). The cytotoxic potential of Formula (I) has been initially evaluated using a panel of cell lines. Similar to the parental drug, Formula (I) demonstrated an excellent safety profile, with a 50% cytotoxic concentration (CC50) of greater than 1000 μM (Coughlin, J. E. et al. Bioorg Med Chem Lett (2010) 20:1783-1786). Formula (II) has been further evaluated for anti-HBV activity in a cell-based assay against wild-type HBV and against lamivudine-(3TC) and adefovir-(ADV) resistant mutant HBV. Formula (II) was found to have antiviral activity against wild-type HBV, with a potency that was in the range of ADV (but less than that of 3TC).

In some embodiments, the method described herein comprises administration of a compound of Formula (I), e.g., Formula (Ia), Formula (Ib), or Formula (Ic), or a pharmaceutically acceptable salt thereof. In other embodiments, the method described herein comprises administration of prodrug of Formula (I) (e.g., a compound of Formula (II), e.g., Formula (IIa), Formula (IIb), or Formula (IIc)) or a pharmaceutically acceptable salt thereof. In other embodiments, the method herein describes administration of a composition comprised of a combination of a compound of Formula (I) (e.g., Formula (Ia), Formula (Ib), or Formula (Ic)) and a compound of Formula (II) (e.g., Formula (Ia), Formula (Ib), or Formula (Ic)) or pharmaceutically acceptable salts thereof. It is well established that the prodrug Formula (I) has been shown to be converted to the active drug Formula (I) (e.g., the Rp- and Sp-Formula (I) isomers) upon administration.

The compounds provided herein may contain one or more asymmetric centers and thus occur as racemates and racemic mixtures, single enantiomers, individual diastereomers and diastereomeric mixtures. All such isomeric forms of these compounds are expressly included within the scope. Unless otherwise indicated when a compound is named or depicted by a structure without specifying the stereochemistry and has one or more chiral centers, it is understood to represent all possible stereoisomers of the compound. The compounds provided herewith may also contain linkages (e.g., carbon-carbon bonds, phosphorus-oxygen bonds, or phosphorus-sulfur bonds) or substituents that can restrict bond rotation, e.g. restriction resulting from the presence of a ring or double bond.

### HBV Infection

The present invention relates to methods for treating a subject infected with HBV through administration of Formula (I) or the prodrug Formula (II), or a pharmaceutically acceptable salt thereof. HBV is an enveloped DNA virus classified as the species type Orthohepadnavirus, which contains three other species, the woodchuck hepatitis virus (WHV), the woolly monkey hepatitis B virus, and the ground squirrel hepatitis virus. The virus is characterized into four major serotypes (adr, adw, ayr, ayw) based upon the antigenic epitopes present on the viral envelope proteins and eight genotypes (genotypes A-H) according to the overall nucleotide sequence of the viral genome. In some embodiments, the methods described herein are used to treat a subject suffering from any known form of HBV infection (e.g., any genotype or serotype of HBV or a combination thereof).

While effective antiviral therapy exists for chronic HBV infection, the infected patient often requires prolonged or lifelong therapy. There are five nucleoside and nucleotide analogs commercially available for treatment of HBV (e.g., lamivudine, adefovir, tenofovir, telbivudine, and entecavir), but their use is limited due to the emergence of drug resistant variants during treatment, the risk of relapse upon treatment discontinuation, and unwarranted side effects. A major challenge of current HBV therapy is to clear the viral, covalently closed circular (ccc) DNA molecule within the nucleus of hepatocytes, which is representing the HBV genome and that is used by the virus as a template for synthesizing the pre-genomic RNA needed for replication. Drugs that target directly HBV cccDNA are currently not available for use in patients. Indirect evidence for treatment-induced reduction of this viral molecule includes the loss of HBV surface antigen (HBsAg), but even after 5 years of therapy with currently available nucleoside and nucleotide analogs, clearance of HBsAg and subsequent seroconversion to antibodies against HBsAg (anti-HBs) are rare events and only achieved in less than 10% of treated patients. In addition, successfully treated patients with antiviral response still exhibit significant levels of HBV-induced liver disease above those in uninfected individuals.

Interferons (e.g., IFN-α) and alternate formulations (e.g., pegylated IFN-α) are also licensed for therapy of HBV but their use is limited because of unwanted side effects. In addition, variability in treatment response of chronic HBV carriers is still a common observation with IFN-α, administered alone or in combination with nucleoside and/or nucleotide analogs, but overall approximately 25-30% of such patients achieve a sustained antiviral response after 2 years of drug administration, including the loss of HBsAg. Therefore, one goal of current HBV therapy is to develop new antiviral compounds that can be used alone or in combination with other anti-HBV drugs to mimic the benefits of IFN-α therapy and to induce suppression of HBV replication, clearance of HBsAg, and seroconversion to anti-HBs in more than one-third of treated patients.

### Pharmaceutical Compositions

The present invention features methods for treating a subject infected with HBV, the methods comprising administering a compound of Formula (I) (e.g., Formula (Ia), Formula (Ib), Formula (Ic), Formula (Id), Formula (Ie), or Formula (If). While it is possible for the compound of the present invention (e.g., a compound of Formula (I)) to be administered alone, it is preferable to administer said compound as a pharmaceutical composition or formulation, where the compounds are combined with one or more pharmaceutically acceptable diluents, excipients or carriers. The compounds according to the invention may be formulated for administration in any convenient way for use in human or veterinary medicine. In certain embodiments, the compounds included in the pharmaceutical preparation may be active itself, or may be a prodrug, e.g., capable of being converted to an active compound in a physiological setting. Regardless of the route of administration selected, the compounds of the present invention, which may be used in a suitable hydrated form, and/or the pharmaceutical compositions of the present invention, are formulated into a pharmaceutically acceptable dosage form such as described below or by other conventional methods known to those of skill in the art.

The amount and concentration of compounds of the present invention (e.g., a compound of Formula (I)) in the pharmaceutical compositions, as well as the quantity of the pharmaceutical composition administered to a subject, can be selected based on clinically relevant factors, such as medically relevant characteristics of the subject (e.g., age, weight, gender, other medical conditions, and the like), the solubility of compounds in the pharmaceutical compositions, the potency and activity of the compounds, and the manner of administration of the pharmaceutical compositions. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

Thus, another aspect of the present invention provides pharmaceutically acceptable compositions comprising a therapeutically effective amount or prophylacticaly effective amount of a compound described herein (e.g., a compound of Formula (I)), formulated together with one or more pharmaceutically acceptable carriers (additives) and/or diluents. As described in detail below, the pharmaceutical compositions of the present invention may be specially formulated for administration in solid or liquid form, including those adapted for oral or parenteral administration, for example, by oral dosage, or by subcutaneous, intramuscular or intravenous injection as, for example, a sterile solution or suspension. However, in certain embodiments the subject compounds may be simply dissolved or suspended in sterile water. In certain embodiments, the pharmaceutical preparation is non-pyrogenic, i.e., does not elevate the body temperature of a patient.

The phrases “systemic administration,” “administered systemically,” “peripheral administration” and “administered peripherally” as used herein mean the administration of the compound other than directly into the central nervous system, such that it enters the patient's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, stabilizing agent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include, but are not limited to: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) ascorbic acid; (17) pyrogen-free water; (18) isotonic saline; (19) Ringer's solution; (20) ethyl alcohol; (21) phosphate buffer solutions; (22) cyclodextrins such as Captisol®; and (23) other non-toxic compatible substances such as antioxidants and antimicrobial agents employed in pharmaceutical formulations.

As set out above, certain embodiments of the compounds described herein may contain a basic functional group, such as an amine, and are thus capable of forming a pharmaceutically acceptable salt with a pharmaceutically acceptable acid. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. In some embodiments, the compound of the present invention is a tartrate salt, e.g., a compound of Formula (Id), Formula (Ie), or Formula (If).

Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutically acceptable carriers, as well as wetting agents, emulsifiers, lubricants, coloring agents, release agents, coating agents, sweetening, flavoring agents, perfuming agents, preservatives, antioxidants, and other additional components may be present in an amount between about 0.001% and 99% of the composition described herein. For example, said pharmaceutically acceptable carriers, as well as wetting agents, emulsifiers, lubricants, coloring agents, release agents, coating agents, sweetening, flavoring agents, perfuming agents, preservatives, antioxidants, and other additional components may be present from about 0.005%, about 0.01%, about 0.05%, about 0.1%, about 0.25%, about 0.5%, about 0.75%, about 1%, about 1.5%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 85%, about 90%, about 95%, or about 99% of the composition described herein.

Pharmaceutical compositions of the present invention may be in a form suitable for oral administration, e.g., a liquid or solid oral dosage form. In some embodiments, the liquid dosage form comprises a suspension, a solution, a linctus, an emulsion, a drink, an elixir, or a syrup. In some embodiments, the solid dosage form comprises a capsule, tablet, powder, dragée, or powder. The pharmaceutical composition may be in unit dosage forms suitable for single administration of precise dosages. Pharmaceutical compositions may comprise, in addition to the compound described herein (e.g., a compound of Formula (I)), a pharmaceutically acceptable carrier, and may optionally further comprise one or more pharmaceutically acceptable excipients, such as, for example, stabilizers (e.g., a binder, e.g., polymer, e.g., a precipitation inhibitor, diluents, binders, and lubricants.

In some embodiments, the composition described herein comprises a liquid dosage form for oral administration, e.g., a solution or suspension. In other embodiments, the composition described herein comprises a solid dosage form for oral administration capable of being directly compressed into a tablet. In addition, said tablet may include other medicinal or pharmaceutical agents, carriers, and or adjuvants. Exemplary pharmaceutical compositions include compressed tablets (e.g., directly compressed tablets), e.g., comprising a compound of the present invention (e.g., a compound of Formula (I)).

Formulations of the present invention include those suitable for parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about 99 percent of active ingredient, preferably from about 5 percent to about 70 percent, most preferably from about 10 percent to about 30 percent. Pharmaceutical compositions of this invention suitable for parenteral administration comprise compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a compound of the present invention (e.g., a compound of Formula (I) or a prodrug thereof (e.g., a compound of Formula (II)), it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered form of the compound of the present invention is accomplished by dissolving or suspending compound in an oil vehicle.

In some embodiments, it may be advantageous to administer the compound of the present invention (e.g., a compound of Formula (I)) in a sustained fashion. It will be appreciated that any formulation that provides a sustained absorption profile may be used. In certain embodiments, sustained absorption may be achieved by combining a compound of the present invention with other pharmaceutically acceptable ingredients, diluents, or carriers that slow its release properties into systemic circulation.

### Routes of Administration

The compounds and compositions used in the methods described herein may be administered to a subject in a variety of forms depending on the selected route of administration, as will be understood by those skilled in the art. Exemplary routes of administration of the compositions used in the methods described herein include topical, enteral, or parenteral applications. Topical applications include but are not limited to epicutaneous, inhalation, enema, eye drops, ear drops, and applications through mucous membranes in the body. Enteral applications include oral administration, rectal administration, vaginal administration, and gastric feeding tubes. Parenteral administration includes intravenous, intraarterial, intracapsular, intraorbital, intracardiac, intradermal, transtracheal, subcuticular, intraarticular, subcapsular, subarachnoid, intraspinal, epidural, intrastemal, intraperitoneal, subcutaneous, intramuscular, transepithelial, nasal, intrapulmonary, intrathecal, rectal, and topical modes of administration. Parenteral administration may be by continuous infusion over a selected period of time. In certain embodiments of the invention, the compositions described herein comprising a compound of Formula (I) (e.g., a compound of Formula (Ia), Formula (Ib), Formula (Ic), Formula (Id), Formula (Ie), or Formula (If)) is administered orally. In other embodiments of the invention, the compositions described herein comprising a compound of Formula (I) (e.g., a compound of Formula (Ia), Formula (Ib), Formula (Ic), Formula (Id), Formula (Ie), or Formula (If)) is administered intravenously.

For intravenous, intraperitoneal, or intrathecal delivery or direct injection, the composition must be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

The choice of the route of administration will depend on whether a local or systemic effect is to be achieved. For example, for local effects, the composition can be formulated for topical administration and applied directly where its action is desired. For systemic, long term effects, the composition can be formulated for enteral administration and given via the digestive tract. For systemic, immediate and/or short term effects, the composition can be formulated for parenteral administration and given by routes other than through the digestive tract.

### Dosages

The compositions of the present invention are formulated into acceptable dosage forms by conventional methods known to those of skill in the art. Actual dosage levels of the active ingredients in the compositions of the present invention (e.g., a compound of Formula (I)) may be varied so as to obtain an amount of the active ingredient which is effective to achieve the desired therapeutic response for a particular subject, composition, and mode of administration, without being toxic to the subject. The selected dosage level will depend upon a variety of pharmacokinetic factors including the activity of the particular compositions of the present invention employed, the route of administration, the time of administration, the rate of absorption of the particular agent being employed, the duration of the treatment, other drugs, substances, and/or materials used in combination with the particular compositions employed, the age, sex, weight, condition, general health and prior medical history of the subject being treated, and like factors well known in the medical arts. A physician or veterinarian having ordinary skill in the art can readily determine and prescribe the effective amount of the composition required. For example, the physician or veterinarian can start doses of the substances of the invention employed in the composition at levels lower than that required in order to achieve the desired therapeutic effect and gradually increase the dosage until the desired effect is achieved. In general, a suitable daily dose of a composition of the invention will be that amount of the substance which is the lowest dose effective to produce a therapeutic effect. Such an effective dose will generally depend upon the factors described above. Preferably, the effective daily dose of a therapeutic composition may be administered as two, three, four, five, six or more sub-doses administered separately at appropriate intervals throughout the day, optionally, in unit dosage forms.

Preferred therapeutic dosage levels are between about 0.1 mg/kg to about 1000 mg/kg (e.g., about 0.2 mg/kg, 0.5 mg/kg, 1.0 mg/kg, 1.5 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 350 mg/kg, 400 mg/kg, 450 mg/kg, 500 mg/kg, 600 mg/kg, 700 mg/kg, 800 mg/kg, 900 mg/kg, or 1000 mg/kg) of the compound or a composition per day administered (e.g., orally or intraperitoneally) to a subject afflicted with a disorder described herein (e.g., HBV infection). Preferred prophylactic dosage levels are between about 0.1 mg/kg to about 1000 mg/kg (e.g., about 0.2 mg/kg, 0.5 mg/kg, 1.0 mg/kg, 1.5 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 350 mg/kg, 400 mg/kg, 450 mg/kg, 500 mg/kg, 600 mg/kg, 700 mg/kg, 800 mg/kg, 900 mg/kg, or 1000 mg/kg) of the compound or a composition per day administered (e.g., orally or intraperitoneally) to a subject. The dose may also be titrated (e.g., the dose may be escalated gradually until signs of toxicity appear, such as headache, diarrhea, or nausea).

The frequency of treatment may also vary. The subject can be treated one or more times per day (e.g., once, twice, three, four or more times) or every so-many hours (e.g., about every 2, 4, 6, 8, 12, or 24 hours). The composition can be administered 1 or 2 times per 24 hours. The time course of treatment may be of varying duration, e.g., for two, three, four, five, six, seven, eight, nine, ten, or more days, two weeks, 1 month, 2 months, 4 months, 6 months, 8 months, 10 months, or more than one year. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

### Patient Selection and Monitoring

The methods of the present invention described herein entail administration of a compound of Formula (I) (e.g., a compound of Formula (Ia), Formula (Ib), Formula (Ic), Formula (Id), Formula (Ie), or Formula (If)) for the treatment of HBV infection. Accordingly, a patient and/or subject can be selected for treatment using a compound of Formula (I) (e.g., a compound of Formula (Ia), Formula (Ib), Formula (Ic), Formula (Id), Formula (Ie), or Formula (If)) by first evaluating the patient and/or subject to determine whether the subject is infected with HBV and determination of the serotypic and genotypic classification of the virus. A subject can be evaluated as infected with HBV using methods known in the art. The subject can also be monitored, for example, subsequent to administration of a compound described herein (e.g., a compound of Formula (I) (e.g., a compound of Formula (Ia), Formula (Ib), Formula (Ic), Formula (Id), Formula (Ie), or Formula (If)) or a pharmaceutically acceptable salt thereof.

In some embodiments, the subject is a mammal. In some embodiments, the subject is a human. In some embodiments, the subject is an adult. In some embodiments, the subject is suffering from an acute form of HBV infection. In some embodiments, the subject is suffering from a chronic form of HBV infection. In some embodiments, the subject has been diagnosed with hepatitis B (e.g., acute or chronic hepatitis B).

In some embodiments, the genotype of the HBV infection is known. In some embodiments, the subject is infected with HBV genotype A (e.g., HBV-A1-7), HBV genotype B (e.g., HBV-B2-5), HBV genotype C (e.g., HBV-C1-16), HBV genotype D (e.g., HBV-D1-7), HBV genotype E, HBV genotype F (e.g., HBV-F1-4), HBV genotype G, HBV genotype H, HBV genotype I, or HBV genotype J. In some embodiments, the subject is infected with HBV genotype A (e.g., HBV-A1-7), HBV genotype B (e.g., HBV-B2-5), HBV genotype C (e.g., HBV-C1-16), HBV genotype D (e.g., HBV-D1-7), HBV genotype E, HBV genotype F (e.g., HBV-F1-4), HBV genotype G, or HBV genotype H. In some embodiments, the subject is infected with HBV genotype A (e.g., HBV-A1-7). In some embodiments, the subject is infected with HBV genotype B (e.g., HBV-B2-5). In some embodiments, the subject is infected with HBV genotype C (e.g., HBV-C1-16). In some embodiments, the subject is infected with HBV genotype D (e.g., HBV-D1-7). In some embodiments, the subject is infected with HBV genotype E. In some embodiments, the subject is infected with HBV genotype F (e.g., HBV-F1-4). In some embodiments, the subject is infected with HBV genotype G. In some embodiments, the subject is infected with HBV genotype H. In some embodiments, the subject is infected with HBV genotype I. In some embodiments, the subject is infected with HBV genotype J.

In some embodiments, the subject is treatment naïve. In some embodiments, the subject has previously been treated for HBV infection. In some embodiments, the subject is suffering from a relapsed HBV infection. In some embodiments, the subject has been treated with an anti-HBV agent other than a compound of Formula (I) and is suffering from a relapsed HBV infection. In some embodiments, the subject has been treated with an interferon, a nucleoside analog, a non-nucleoside antiviral, or an immune enhancer and is suffering from a relapsed HBV infection. In some embodiments, the subject has been treated with an interferon, e.g., peg-interferon alfa (e.g., peg-interferon alfa-2a or peg-interferon alfa-2b) and is suffering from a relapsed HBV infection. In some embodiments, the subject has been treated with ribavirin and is suffering from a relapsed HBV infection. In some embodiments, the subject has been treated with a nucleoside analog, e.g., lamivudine, adefovir dipivoxil, entecavir, telbivudine, clevudine, ribavarin, tenofovir, tenofovir alafenamide, besifovir, or AGX-1009, and is suffering from a relapsed HBV infection. In some embodiments, the subject has been treated with a non-nucleoside antiviral agent, e.g., NOV-225, BAM 205, Myrcludex B, ARC-520, BAY 41-4109, REP 9AC, Alinia (nitazoxanide), Dd-RNAi, NVR-121 (NVR 3-778), BSBI-25, NVP-018, TKM-HBV, or ALN-HBV, and is suffering from a relapsed HBV infection. In some embodiments, the subject has been treated with a immune enhancer, e.g., zadaxin (thymosin alpha-1), GS-4774, CYT107 (interleukin-7), Dv-601, HBV core antigen vaccine, or GS-9620, and is suffering from a relapsed HBV infection.

In some embodiments, the subject has been diagnosed with cirrhosis of the liver. In some embodiments, the subject has been diagnosed with hepatocellular carcinoma. In some embodiments, the subject has been diagnosed with hepatocellular carcinoma and is awaiting liver transplantation.

In some embodiments, the subject has been further diagnosed with an HIV infection. In some embodiments, the strain of HIV infection is known. In some embodiments, the subject is infected with HIV-1 or HIV-2 (e.g., strain 1 or strain 2).

In some embodiments, the subject has been diagnosed with hepatitis B (e.g., acute or chronic hepatitis B, e.g., a resistant variant of acute or chronic hepatitis B).

### Combination Therapies

In some embodiments, additional therapeutic agents may be administered with compositions of the present invention for the treatment of HBV or any symptom or associated condition thereof. When combination therapy is employed, the additional therapeutic agent(s) can be administered as a separate formulation or may be combined with any of the compositions described herein.

For example, any of the methods described herein may further comprise the administration of a therapeutically effective amount of an additional agent in conjunction with a compound of Formula (I) (e.g., a compound of Formula (Ia), Formula (Ib), Formula (Ic), Formula (Id), Formula (Ie), or Formula (If)). In some embodiments, the additional agent is an antiviral agent or an anticancer agent. In some embodiments, the antiviral agent comprises an interferon, a nucleoside analog, a non-nucleoside antiviral, or a non-interferon immune enhancer. In some embodiments, the interferon comprises interferon alfa-2a, interferon alfa-2b, interferon alfa-n1, interferon alfacon-1, or a pegylated interferon (e.g., peginterferon alfa-2a, peginterferon alfa-2b). In some embodiments, the nucleoside analog comprises lamivudine, adefovir dipivoxil, entecavir, telbivudine, clevudine, ribavarin, tenofovir, tenofovir dipivoxil, tenofovir alafenamide, besifovir, or AGX-1009. In some embodiments, the antiviral agent is entecavir. In some embodiments, the antiviral agent is tenofovir (e.g., tenofovir dipivoxil or tenofovir alafenamide). In some embodiments, the antiviral compound comprises NOV-225, BAM 205, Myrcludex B, ARC-520, BAY 41-4109, REP 9AC, Alinia (nitazoxanide), Dd-RNAi, NVR-121 (NVR 3-778), BSBI-25, NVP-018, TKM-HBV, or ALN-HBV. In some embodiments, the non-interferon immune enhancer comprises zadaxin (thymosin alpha-1), GS-4774, CYT107 (interleukin-7), Dv-601, HBV core antigen vaccine, or GS-9620. In some embodiments, the antiviral agent is a capsid inhibitor, an entry inhibitor, a secretion inhibitor, a microRNA, an antisense RNA agent, an RNAi agent, or other agent designed to inhibit viral RNA. In some embodiments, the anticancer agent is selected from methotrexate, 5-fluorouracil, doxorubicin, vincristine, bleomycin, vinblastine, dacarbazine, toposide, cisplatin, epirubicin, and sorafenib tosylate.

Administration in combination can proceed by any technique apparent to those of skill in the art including, for example, separate, sequential, concurrent, and alternating administration. As used herein, “administered in combination” or a combined administration of two or more agents means that two or more agents (e.g., compounds described herein) are administered to a subject at the same time or within an interval such that there is overlap of an effect of each agent on the patient. Preferably they are administered within 15, 10, 5, or 1 minute of one another. In some embodiments, the combination of a compound of Formula (I) and the additional agent has a synergistic or additive effect. In some embodiments, the term “additive” refers to an outcome wherein when two agents are used in combination, the combination of the agents acts in a manner equal to but not greater than the sum of the individual anti-HBV activities of each agent.

In some embodiments, the terms “synergy” or “synergistic” refer to an outcome wherein when two agents are used in combination, the combination of the agents acts so as to require a lower concentration of each individual agent than the concentration required to be efficacious in the absence of the other agent. In some embodiments, a synergistic effect results in a reduced in a reduced minimum inhibitory concentration of one or both agents, such that the effect is greater than the sum of the effects. A synergistic effect is greater than an additive effect. In some embodiments, the agents in the composition herein may exhibit a synergistic effect, wherein the anti-HBV activity at a particular concentration is greater than at least about 1.25, 1.5, 1.75, 2, 2.5, 3, 4, 5, 10, 12, 15, 20, 25, 50, or 100 times the anti-HBV activity activity of either agent alone. Preferably the administrations of the agents are spaced sufficiently close together such that a combinatorial (e.g., a synergistic) effect is achieved.

The combinations can have synergistic effect when used to treat a subject suffering from an HBV infection. The agents can be administered simultaneously, for example in a combined unit dose (providing simultaneous delivery of both agents). Alternatively, the agents can be administered at a specified time interval, for example, an interval of minutes, hours, days or weeks. Generally, the agents are concurrently bioavailable, e.g., detectable, in the subject.

### EXAMPLES

### Example 1. Synthesis of Exemplary Compound of the Invention

### Synthesis of Formula (Ie) and Formula (If)

2.0 g (2.842 mmol) of either the Rp or Sp isomer (Formula (IIb) or Formula (IIc) was weighed out in a 100 mL 1N round bottom flask containing a stir bar. (L)-(+)-tartaric acid (427 mg; 2.842 mmol; 1.0 equiv.) was weighed and added to the vial. Acetonitrile (20 mL) followed by HPLC grade water (20 mL) was added, and the flask was capped and the mixture was stirred to give a clear, colorless solution. The clear, colorless solution was stirred for 6 d at room temperature. After 64 h of stirring time, additional HPLC grade water (20 mL) was added and the clear solution was stirred for another 6 d. Once the stirring was stopped, the solvent was carefully evaporated in vacuo to obtain an aqueous slightly cloudy solution. The slightly cloudy solution was passed through a cotton plug. The clear filtrate obtained was transferred to a 500 mL 1N pear-shaped flask, freeze-dried and lyophilized to yield either 2.382 g of Formula (Ie) or 2.387 g of Formula (If), respectively.

### Example 2. Efficacy of Exemplary Compounds for Treating HBV Infection

The goal of this study was to evaluate the efficacy of exemplary compounds for the treatment of HBV infection in transgenic mice. Homozygous male transgenic HBV mice (21.6±2.8 g) originally obtained from the laboratory of Dr. Frank Chisari (Scripps Research Institute, LaJolla, Calif.) and derived from founder 1.3.32 were used in this study. Female and male HBV transgenic mice were block-randomized to the treatment groups (Table 1). Compounds of Formula (Ie) and Formula (If) were administered at 0.1 mL/30-gram mouse by oral gavage. Adefovir dipivoxil (ADV) was prepared as a solution of 2 mg/mL in 0.025 M sodium citrate, wherein 0.1 mL was administered by oral gavage (per os, p.o.) per 30 g mouse for a dosage of 10 mg/kg/day.

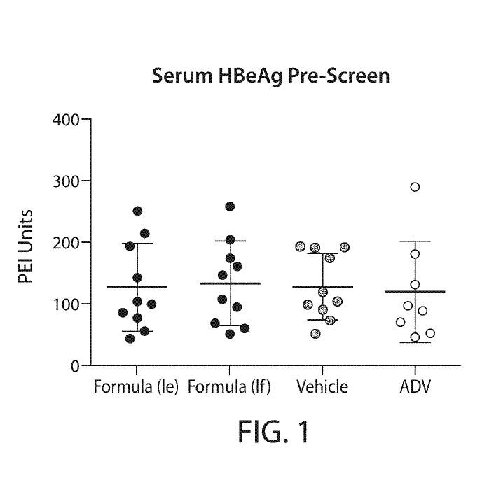

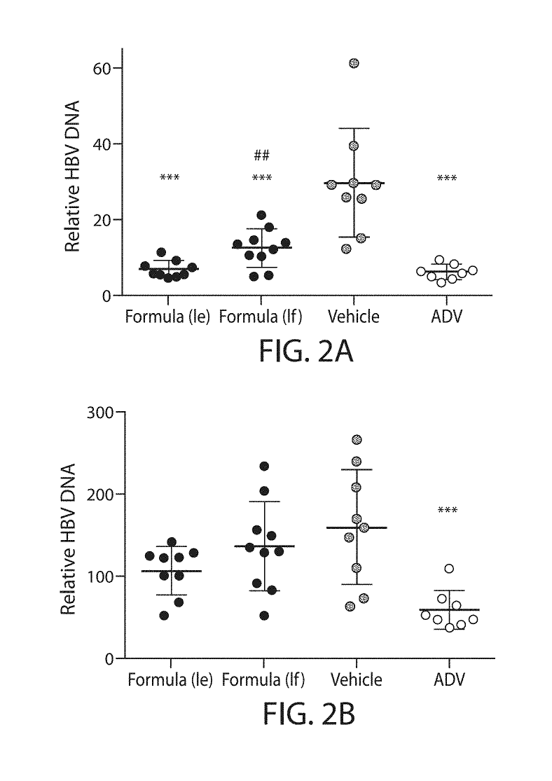

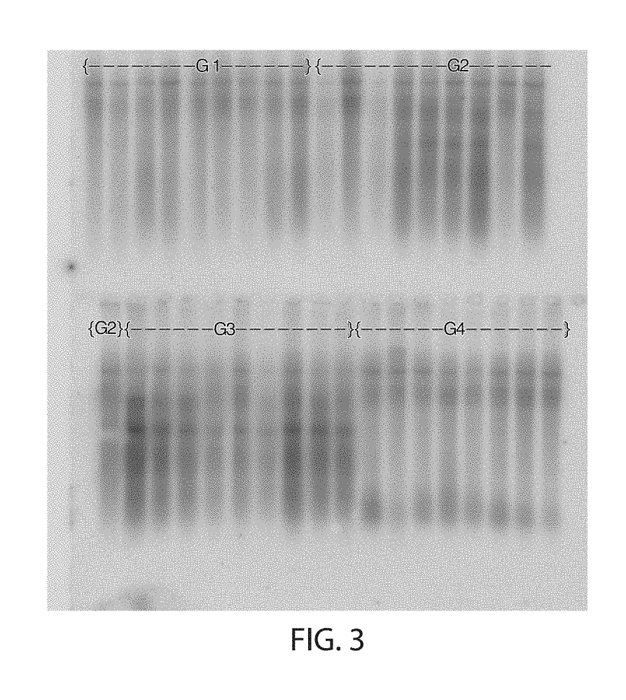

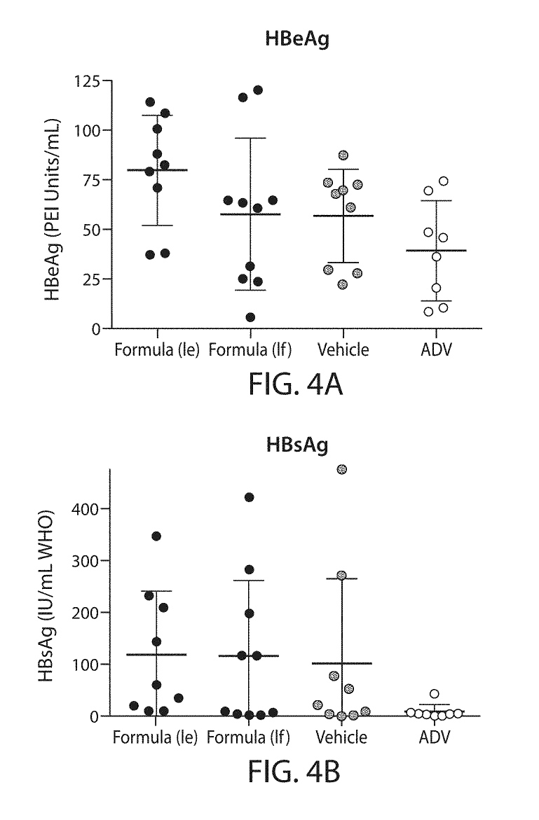

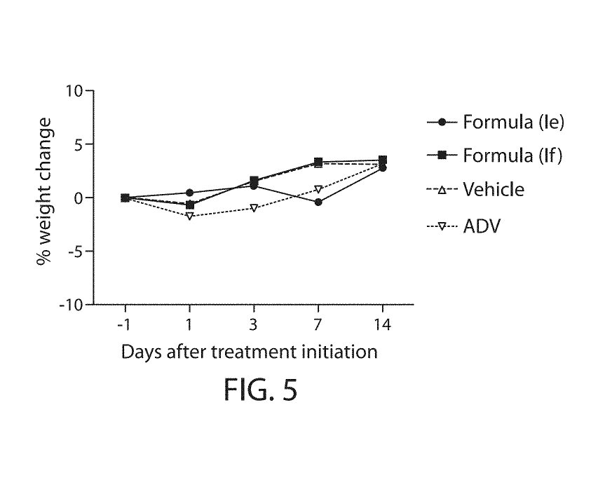

Treatment was initiated at day 1. After the last treatment on day 14, necropsy was performed to obtain tissues to assay liver HBV DNA and sera for HBV antigens HBeAg and HBsAg. Weights were obtained on days −1, 1 (day of treatment initiation), 3, 7, and 14. Data were analyzed by one-way analysis of variance. Two animals died due to oral gavage treatment at day 7, one in group 1 and another in group 3. The starting levels of HBeAg in the subjects tested are summarized in FIG. 1. FIGS. 2A-2B show the effect of Formula (Ie) and Formula (If) on liver HBV DNA levels using Southern blot hybridization (FIG. 2A) and semi-quantitative PCR (FIG. 2B) as compared to vehicle and ADV. As shown, both Formula (Ie) and Formula (If) result in lowered HBV DNA levels in the liver, which is further demonstrated in FIG. 3. Neither Formula (Ie) or Formula (If) had an effect on serum levels of HBeAg or HBsAg (FIGS. 4A-4B) nor on whole body weight of the mice (FIG. 5) relative to vehicle values.

TABLE 1
Experimental design

| | | | | Treatment |
|---|---|---|---|---|
| #/Cage | Group # | Compound | Dosage | Schedule |
| 10 | 1 | Formula (Ie) | high | per os, qd X 14 d |
| 10 | 2 | Formula (If) | high | per os, qd X 14 d |
| 10 | 3 | vehicle | — | per os, qd X 14 d |
| 8 | 4 | adefovir dipivoxil | 10 mg/kg | per os, qd X 14 d |

### Equivalents

The disclosures of each and every patent, patent application, and publication cited herein are hereby incorporated herein by reference in their entirety. While this disclosure has been described with reference to specific aspects, it is apparent that other aspects and variations may be devised by others skilled in the art without departing from the true spirit and scope of the disclosure. The appended claims are intended to be construed to include all such aspects and equivalent variations. Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference.

While this disclosure has been particularly shown and described with references to preferred embodiments thereof, it will be understood by those skilled in the art that various changes in form and details may be made therein without departing from the scope of the disclosure encompassed by the appended claims.

1. A method of treating a subject infected with the Hepatitis B virus, the method comprising administering to the subject a salt form of a compound of Formula (I), wherein the compound is selected from: 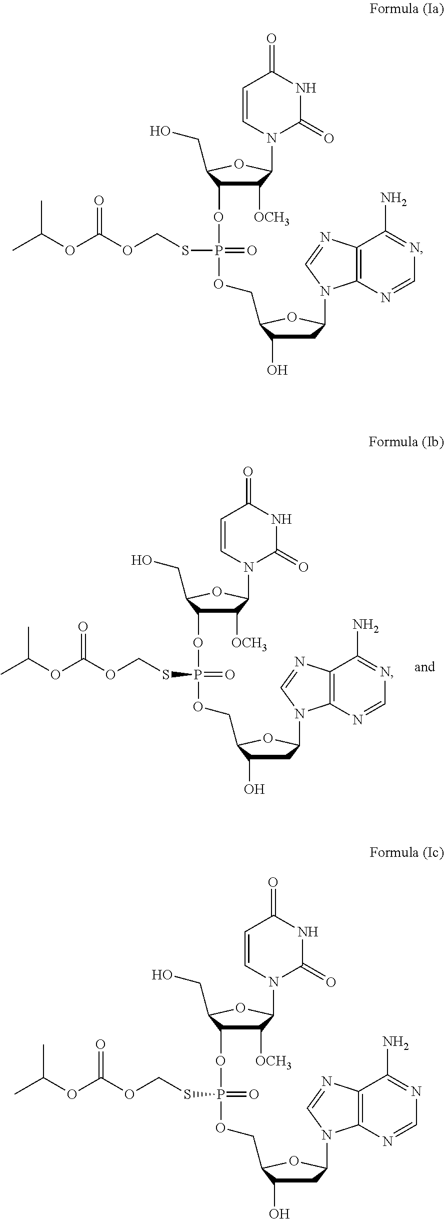
to thereby treat the subject.

2. The method of claim 1, wherein the salt form of a compound of Formula (I) is a tartrate salt.

3. The method of claim 1, wherein the compound is administered to the subject as a composition (e.g., a pharmaceutical composition).

4. The method of claim 3, wherein the composition comprises a pharmaceutically acceptable excipient.

5. The method of claim 4, wherein the composition comprises a mixture of compounds of Formula (I), e.g., Formula (Ib) and Formula (Ic).

6. The method of claim 5, wherein the composition comprises Formula (Ib) and comprises less than about 5% of Formula (Ic) (e.g., less than about 4%, less than about 3%, less than about 2%, less than about 1%, less than about 0.5%, or less than about 0.1% of Formula (Ic)), or is substantially free of Formula (Ic).

7. The method of claim 5, wherein the composition comprises Formula (Ic) and comprises less than about 5% of Formula (Ib) (e.g., less than about 4%, less than about 3%, less than about 2%, less than about 1%, less than about 0.5%, or less than about 0.1% of Formula (Ib), or is substantially free of Formula (Ib)).

8. The method of claim 1, wherein the compound of Formula (I) is a compound selected from:

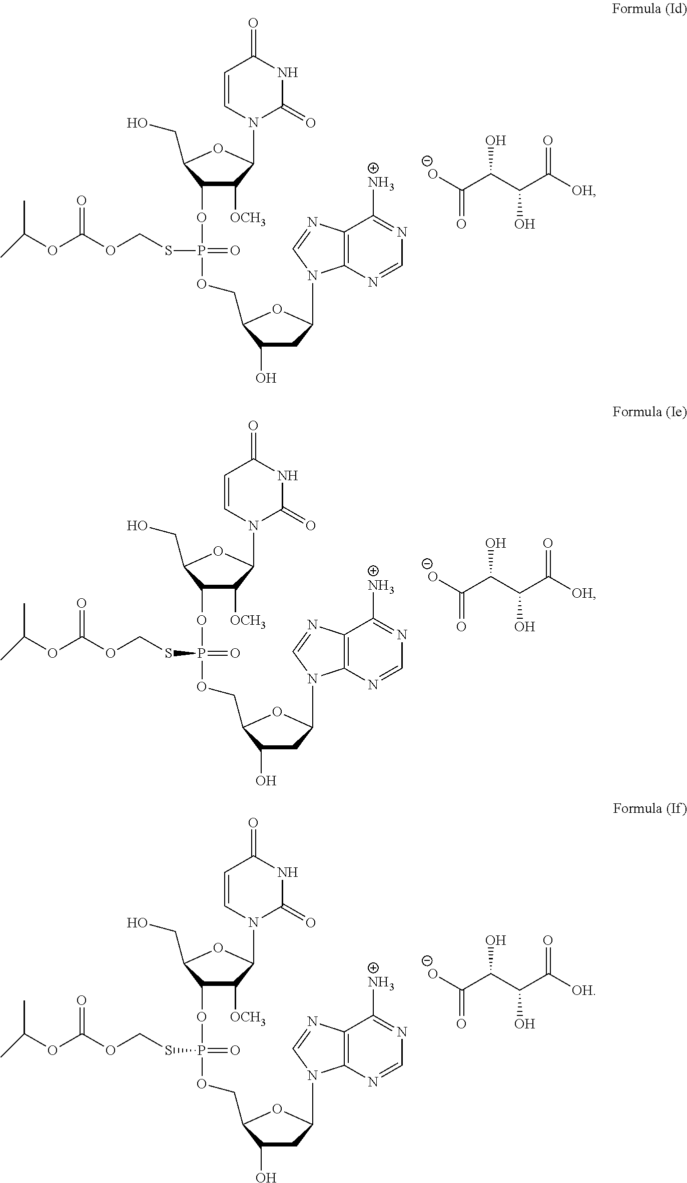

9. The method of claim 8, wherein the compound is administered to the subject as a composition comprising a mixture of compounds of Formula (I), e.g., Formula (Ie) and Formula (If).

10. The method of claim 9, wherein the composition comprises Formula (Ie) and comprises less than about 5% of Formula (If) (e.g., less than about 4%, less than about 3%, less than about 2%, less than about 1%, less than about 0.5%, or less than about 0.1% of Formula (If), or is substantially free of Formula (If)).

11. The method of claim 9, wherein the composition comprises Formula (If) and comprises less than about 5% of Formula (Ie) (e.g., less than about 4%, less than about 3%, less than about 2%, less than about 1%, less than about 0.5%, or less than about 0.1% of Formula (Ie), or is substantially free of Formula (Ie)).

12. The method of claim 1, wherein the compound of Formula (I) is administered orally.

13. The method of claim 1, wherein the compound of Formula (I) is administered parenterally.

14. The method of claim 1, wherein the subject is a mammal.

15. The method of claim 1, wherein the subject is a human.

16. The method of claim 1, wherein the method comprises daily administration.

17. The method of claim 16, wherein the administration is once daily.

18. The method of claim 1, wherein the dosage comprises about 0.5 mg/kg to about 50 mg/kg.

19. The method of claim 18, wherein the dosage comprises 5 mg/kg to about 50 mg/kg.

20. The method of claim 4, wherein the composition comprises a liquid or a solid dosage form.

21. The method of claim 20, wherein the liquid dosage form comprises a suspension, a solution, a linctus, an emulsion, a drink, an elixir, or a syrup.

22. The method of claim 21, wherein the solid dosage form comprises a capsule, tablet, dragée, or powder.

23. The method of claim 1, further comprising the administration of a therapeutically effective amount of an additional agent.

24. The method of claim 23, wherein the additional agent is an antiviral agent or an anticancer agent.

25. The method of claim 24, wherein the antiviral agent comprises an interferon, a nucleoside analog, a non-nucleoside antiviral, or a non-interferon immune enhancer.

26. The method of claim 1, further comprising analyzing or receiving analysis of the body weight and temperature of the subject at least once a week until the end of treatment.

27. The method of claim 1, further comprising analyzing or receiving analysis of a blood sample from the subject at least once prior to the end of treatment.

28. The method of claim 27, wherein the blood sample is analyzed for viral load, e antigen level (e.g., HBeAg level), surface antigen level (HBsAg), or core antigen level (e.g., HBCrAg).

29. The method of claim 27, wherein the blood sample is analyzed for the expression level of interferon (e.g., interferon alfa or interferon beta), an interferon stimulating protein (e.g., ISG15, CXCL10, OAS 1), or other cytokine.

30. The method of claim 1, further comprising analyzing or receiving analysis of a liver biopsy specimen from the subject at least once prior to the end of treatment.

31. The method of claim 30, wherein the liver biopsy specimen is analyzed for the level of viral DNA, viral RNA, viral antigens, or cccDNA.

32. The method of claim 30, wherein the liver biopsy specimen is analyzed for the expression level of interferon (e.g., interferon alfa or interferon beta), an interferon stimulating protein (e.g., ISG15, CXCL10, OAS 1), or other cytokine.

33. The method of claim 30, wherein the liver biopsy specimen is analyzed for the expression of a pattern recognition receptor.

34. The method of claim 33, wherein the pattern recognition receptor comprises RIG-I, NOD2, or STING.

35. The method of claim 30, wherein the liver biopsy specimen is analyzed for the reduction of liver inflammation, necrosis, steatosis, or fibrosis.
